(19) United States
(12) Patent Application Publication
(10) Pub. No.: US 2021/0073982 A1
(43) Pub. Date: Mar. 11, 2021

(54) MEDICAL IMAGE PROCESSING METHOD AND APPARATUS, ELECTRONIC DEVICE, AND STORAGE MEDIUM

(71) Applicant: BEIJING SENSETIME TECHNOLOGY DEVELOPMENT CO., LTD., Beijing (CN)

(72) Inventors: Qing XIA, Beijing (CN); Yunhe GAO, Beijing (CN)

(21) Appl. No.: 16/953,896

(22) Filed: Nov. 20, 2020

Related U.S. Application Data
(63) Continuation of application No. PCT/CN2018/117759, filed on Nov. 27, 2018.

(30) Foreign Application Priority Data
Jul. 21, 2018 (CN) 201810818690.X

(51) Int. Cl. G06T 7/00; G06T 7/70; G06T 7/11; G16H 30/20; G06N 3/08

(52) U.S. Cl. CPC G06T 7/0012; G06T 7/70; G06T 7/11; G06T 2207/30012; G06N 3/08; G06T 2207/20084; G06T 2207/20081; G16H 30/20

(57) ABSTRACT

A medical image processing method and apparatus, an electronic device, and a storage medium are disclosed. The method includes: detecting a medical image by using a first detection module to obtain first position information of a first target in a second target, wherein the second target comprises at least two of the first targets; segmenting the second target by using the first detection module according to the first position information to obtain a target feature map and first diagnostic auxiliary information of the first target.

### CROSS-REFERENCE TO RELATED APPLICATIONS

The present disclosure is a U.S. continuation application of International Application No. PCT/CN2018/117759, filed on Nov. 27, 2018, which claims priority to Chinese Patent Application No. 201810818690.X, filed with the Chinese Patent Office on Jul. 24, 2018. The disclosures of International Application No. PCT/CN2018/117759 and Chinese Patent Application No. 201810818690.X are incorporated herein by reference in their entireties.

### BACKGROUND

A medical image is important auxiliary information for helping a doctor in diagnosis. In the related art, the doctor reads an image by holding a physical image of the medical image or on a computer for diagnosis after the medical image is photographed. However, the medical image generally relates to non-surface structures photographed by means of various rays and the like, it is confined to a photographing technology or a photographing scene, some angles may not be visible, and the diagnosis of a medical personnel may be affected.

### SUMMARY

The present disclosure relates to, but is not limited to, the technical field of information, and in particular, to a medical image processing method and apparatus, an electronic device, and a storage medium.

Embodiments of the present disclosure are expected to provide a medical image processing method and apparatus, an electronic device, and a storage medium.

The technical solutions of the present disclosure are implemented in the following manners: in a first aspect, the embodiments of the present disclosure provide a medical image processing method, including:

detecting a medical image by using a first detection module, and obtaining first position information of a first target in a second target, wherein the second target includes at least two of the first targets;

segmenting the second target by using the first detection module according to the first position information to obtain a target feature map of the first target and a first diagnostic auxiliary information of the first target.

In a second aspect, the embodiments of the present disclosure provide a medical image processing apparatus, including:

a first detection unit, configured to detect a medical image by using a first detection module to obtain first position information of a first target in a second target, wherein the second target includes at least two of the first targets;

a processing unit, configured to segment the second target by using the first detection module according to the first position information to obtain a target feature map of the first target and a first diagnostic auxiliary information of the first target.

In a third aspect, the embodiments of the present disclosure provide a non-transitory computer storage medium, and configured to store computer-readable instructions; where execution of the instructions by the processor causes the processor to perform the technical solution in the first aspect.

In a fourth aspect, the embodiments of the present disclosure provide a computer program product, and the program product includes computer executable instructions; after the computer executable instructions are executed, the method provided according to the technical solution in the first aspect can be implemented.

In a fifth aspect, the embodiments of the present disclosure provide an image processing device, including:

a memory, configured to store information;

a processor, connected to the memory, and configured to execute computer executable instructions stored on the memory to implement the method provided according to the technical solution in the first aspect.

### BRIEF DESCRIPTION OF DRAWINGS

FIG. 1 is a schematic flowchart of a first medical image processing method provided by embodiments of the present disclosure;

FIG. 2 is a schematic flowchart of a second medical image processing method provided by embodiments of the present disclosure;

FIG. 3 is a schematic flowchart of a third medical image processing method provided by embodiments of the present disclosure;

FIG. 4 is a schematic change diagram from a medical image to a segmented image provided by embodiments of the present disclosure;

FIG. 5 is a schematic structural diagram of a medical image processing apparatus provided by embodiments of the present disclosure; and

FIG. 6 is a schematic structural diagram of a medical image processing device provided by embodiments of the present disclosure.

### DETAILED DESCRIPTION

A medical image is important auxiliary information for helping a doctor in diagnosis. However, how to provide comprehensive, complete, and effective information to the medical personnel is a problem to be further solved in the related art.

According to the technical solutions provided in the embodiments of the present disclosure, a medical model is detected by using the first detection module, the first target is wholly separated from the second target where the first target is; in this way, on the one hand, the number of times of viewing the first target by a doctor only in the second target is reduced, so that the doctor can more comprehensively and completely view the first target; on the other hand, the embodiments of the present disclosure provide an output target feature map, the target feature map includes features, which is configured to a medical diagnosis, of the first target, and thus the interference on unnecessary interference features is removed, and diagnosis interference is reduced; according to yet another aspect, the first diagnostic auxiliary information is further generated to provide more assistance to the diagnosis of the medical personnel. In this way, according to the medical image processing method in the embodiments, a more comprehensive and complete target feature image reflecting a medical diagnosis first target can be obtained and the first diagnostic auxiliary information is provided to facilitating diagnosis.

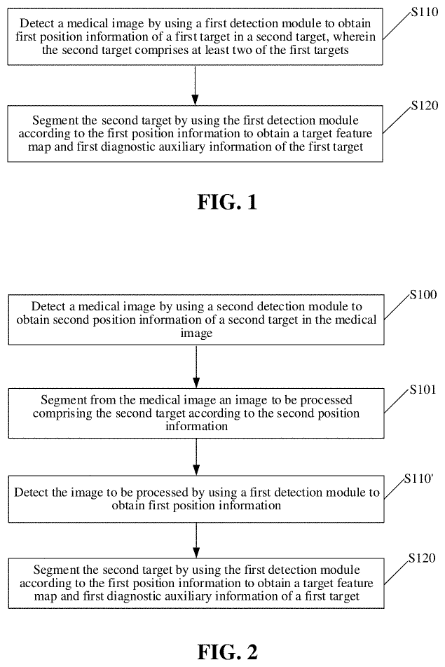

As shown in FIG. 1, the embodiments provide a medical image processing method, and the method includes the following operations.

At operation S110, a medical image is detected by using a first detection module to obtain first position information of a first target in a second target, wherein the second target includes at least two of the first targets;

At operation S120, the second target is segmented by using the first detection module according to the first position information to obtain a target feature map and first diagnostic auxiliary information of the first target.

The first detection module may be different modules having a detection function. For example, the first detection module may be functional modules corresponding to different data models. The data models include: different deep learning models. The deep learning models include: a neural network model, a vector machine model and the like, but are not limited to the neural network model or the vector machine.

The medical image may be image information photographed in medical diagnostic processes, for example, a nuclear magnetic resonance image, for another example, a Computed Tomography (CT) image.

The first detection module may be the neural network model and the like, and feature extraction of the second target can be performed in the neural network model by means of processing such as convolution to obtain the target feature map and generate the first diagnostic auxiliary information.

The medical image in some embodiments may include: a Dixon sequence; the Dixon sequence includes multiple two-dimensional images acquired for the same acquired object in different acquired angles; and the two-dimensional images can be configured to constructing a three-dimensional image for a first acquired object.

The first position information may include: information for describing the position of the first target located in the second target, and the position information specifically may include: a coordinate value of the first target in an image coordinate, for example, an edge coordinate value of a first target edge, a central coordinate value of a first target center, and a size value of the first target in the second target in different scales.

The first target is the ultimate target of diagnosis, and the second target may include multiple first targets. For example, in some embodiments, the second target may be a vertebral column, and the first target may be an intervertebral disc between vertebras or adjacent vertebras. In some other embodiments, the second target further may be a rib-sternum of a chest; moreover, the rib-sternum may consist of multiple J-shaped ribs. The first target may be a single rib in the rib-sternum.

In conclusion, the second target and the first target may be different objects that need medical diagnoses, but are not limited to the aforementioned examples.

In operation S120, image processing can be performed on the medical image by using the first detection module to segment the second target, so that the target feature maps of the first targets constituting the second target are separated, and the first diagnostic auxiliary information, which is included in the corresponding target feature map, of the first target is obtained.

In some embodiments, the target feature map may include: an image which is cut out from the original medical image and include a single first target.

In some other embodiments, the target feature map may further include: a feature map which is regenerated based on the original medical image and represents target features. The feature map includes different diagnostic information in which the medical diagnoses are required; moreover, detail information not related to the medical diagnoses is removed. For example, taking the intervertebral disc as an example, target features of the outer contour, the shape and volume of the intervertebral disc related to the medical diagnoses, but some texture on the surface of the intervertebral disc is not related to a medical treatment, at this moment, the target feature map may only include: information of the outer contour, the shape and volume and the like of the intervertebral disc related to the medical diagnoses; moreover, interference features such as surface texture not related to the medical diagnoses are removed. After the target feature map is output, when a medical personnel diagnoses based on the target feature map, because interference is reduced, fast and accurate diagnoses can be achieved.

The first diagnostic auxiliary information may be information of attributes or states of the first target in the target feature map corresponding to descriptions. The first diagnostic auxiliary information may be information directly attached to the target feature map, and may also be information stored into the same file as the target feature map.

For example, in operation S120, the first detection module generates a diagnostic file including the target feature map, the diagnostic file may be a three-dimensional dynamic image file; when playing back the three-dimensional dynamic file, an angle currently displayed in a three-dimensional target feature map can be adjusted by means of specific software, and the first diagnostic auxiliary information is displayed in a display window, so that the medical personnel such as a doctor can see the first diagnostic auxiliary information while viewing the target feature map, thereby facilitating the medical personnel diagnosing by combining the target feature map and the first diagnostic auxiliary information.

The three-dimensional target feature map may be formed by constructing multiple two-dimensional target feature maps. For example, operations of operation S110 to operation S120 are performed on each two-dimensional image in the Dixon sequence, so that at least one target feature map is generated according to one two-dimensional image; multiple target feature maps are generated according to multiple two-dimensional images, and three-dimensional target features of the first target can be constructed for the target feature maps, corresponding to different acquired angles, of the same first target.

In some embodiments, the target feature map output in operation S120 may also be the three-dimensional target feature map in which a three-dimensional construction is directly completed.

The type of the first diagnostic auxiliary information may include:

text information, for example, performing an attribute description in a text form;

marking information, for example, marking the sizes of different dimensions (directions) of the first target such as the intervertebral disc by combining auxiliary information such as coordinate axes on the coordinate axes by means of arrow and text descriptions and the like.

In the embodiment, image pixels of the target feature map may maintain consistency with pixels of an image to be processed, for example, the image to be processed is an image including N*M pixels, and the target feature map may also be a target feature map including N*M pixels.

In some embodiments, if the second target includes F first targets, F three-dimensional target feature maps can be output, or F groups of two-dimensional target features are output; one group of two-dimensional target feature maps correspond to one first target, and the three-dimensional target feature maps of the first target can be constructed.

In some embodiments, the target feature map and the first diagnostic auxiliary information as two portions of information form a target feature file to be output, for example, the first diagnostic auxiliary information is stored in the target feature file in a text information form; and the target feature map is stored in the target file in a picture form.

In some other embodiments, the first diagnostic auxiliary information is attached onto the target feature map to form a diagnostic image; in this way, the first diagnostic auxiliary information and the target feature map are a portion of the diagnostic image, and are stored in an image information form.

The operation S120 may include the following operation: a pixel-level segmentation is performed on the second target by using the first detection module according to the first position information to obtain the target feature map and the first diagnostic auxiliary information.

In the embodiment, the pixel-level segmentation is performed on the second target in the medical image by using the second detection module, so that different first targets can be completely separated and the boundaries clearly identified, thereby facilitating the doctor diagnosing according to the segmented target feature maps and/or first diagnostic auxiliary information.

Similarly, a second detection module may also be different functional modules that can implement the segmentation of the second target. For example, the second detection module may also be: functional modules running different data models; for example, running modules of different deep learning models.

The pixel-level segmentation indicates that segmentation accuracy reaches pixel accuracy, for example, different intervertebral discs are separated in the image, or when the separation of the intervertebral disc and vertebral column are performed in the image, it can be accurate to a certain pixel, and whether the pixel belongs to the intervertebral disc or the vertebral column is specifically determined; but not using a pixel region formed by multiple pixels as the segmentation accuracy. Therefore, the first target can be accurately separated from the second target, thereby facilitating accurate diagnosis.

As shown in FIG. 2, the method further includes the following operations.

At operation S100, the medical image is detected by using the second detection module to obtain second position information of the second target in the medical image;

At operation S101, an image to be processed including the second target is segmented from the medical image according to the second position information;

The operation S110 may include operation S110′: the image to be processed is detected by using the first detection module to obtain the first position information.

In the embodiment, the second detection module may pre-process the medical image, thereby facilitating subsequently segmenting by the first detection module the image to be processed from the medical image.

In the embodiment, the second detection module may be the neural network model, at least outer contour information and the like of the second target can be obtained by means of convolution processing and the like in the neural network model, and the second position information is obtained based on the outer contour information. In this way, background information and interference information unrelated to the diagnosis are cut out in the image to be processed with respect to the original medical image.

The background information may be image information of a blank image region, in the medical image, without carrying the amount of information

The interference information may be image information except for that of the second target. For example, the medical image may be a nuclear magnetic resonance image for a human waist; human waist information is acquired in the nuclear magnetic resonance image, and information such as a tissue, a lumbar vertebra, and ribs information of the waist is acquired. If the second target is the lumbar vertebra, image information corresponding to the tissue and ribs is the interference information.

In operation S100, each two-dimensional image can be detected by using the second detection module to determine the second position information.

The second position information may include: a coordinate value of an image region where the second target is in an image coordinate, for example, coordinate values of the outer contour of the second target in the two-dimensional images. The coordinate value may be an edge coordinate value of a second target edge, or the size of the second target, and a central coordinate value of a second target center. The second position information may be different information in which the second target can be localized from an image, but is not limited to the coordinate value. For another example, the image is detected by using different detection frames, and the second position information may further be an identification of the detection frame. For example, one image may be covered by several detection frames without overlapping and intervals; if the second target is within a Tth detection frame, the identification of the Tth detection frame is one of the second position information. In conclusion, the second position information includes multiple forms, is not limited to the coordinate value and not limited to a frame identification of the detection frame, either.

After the determination of the second position information is completed by using the second detection module, the image to be processed that needs to be processed by the first detection module is segmented from the original medical image according to the second position information, and the segmentation of the image to be processed can be processed by the second detection module, also can be processed by the first detection module, and even can be processed by a third sub-model located between the second detection module and the first detection module.

The image to be processed is an image in which the background information and the interference information are removed and the second target is included. Obtaining the image to be processed by processing the original medical image can greatly reduce the calculation and improve processing speed with respect to directly performing segmentation processing of the second target on the original medical image in the related art; moreover, the problem that the subsequent extraction of the target feature map and the first diagnostic auxiliary information is inaccurate caused due to the introduction of the background information and the interference information is reduced, and the accuracy of the target feature map and the first diagnostic auxiliary information is improved.

The image processing is only required to be performed on the image to be processed by using the first detection module, the second target can be segmented, so that the first targets constituting the second target are separated from the original medical image, and then the first diagnostic auxiliary information of the first target included in the corresponding target feature map is obtained by processing the separated medical image.

In some embodiments, as shown in FIG. 3, operation S110 may include the following operations.

At operation S111, the image to be processed or the medical image is detected by using the first detection module to obtain an image detection region of the first target;

at operation S112, the image detection region is detected to obtain the outer contour information of the second target;

at operation S113, a mask region is generated according to the outer contour information.

at operation S114, a segmented image including the second target is segmented from the medical image or the image to be processed according to the mask region.

For example, the medical image or the image to be processed is segmented by using the detection frame to obtain the image detection region where the first target is.

The extraction of the outer contour information of the second target is performed on the image detection region, for example, image processing is performed on the image detection region by means of a convolutional network of which the outer contour can be extracted, so as to obtain the outer contour information, and the mask region can be generated by extracting the outer contour information. The mask region may be information in the forms of just covering a matrix or a vector of the first target and the like. The mask region is located in the image detection region, and the area of the mask region is generally less than the area of the image detection region. The image detection region may be a standard rectangular region; and the region corresponding to the mask region may be an irregular region. The shape of the mask region is determined by the outer contour of the first target.

In some embodiments, the segmented image can be extracted from the image to be processed or the medical image by means of related calculation of the mask region and the medical image. For example, a transparent mask region is added onto an all-black image to obtain an image having a region to be transparent, after the image is overlapped with the corresponding image to be processed or the medical image, the segmented image only including the second target is generated. Or the all-black region is cut out from the overlapped image to obtain the segmented image. For another example, an all-white image adds a transparent mask region to obtain an image having a region to be transparent, after the image is overlapped with the corresponding medical image, the segmented image only including the second target is generated. Or the all-white region is cut out from the overlapped image to obtain the segmented image. For another example, the corresponding segmented image is directly extracted from the medical image directly based on a pixel coordinate of each pixel where the mask region is.

Definitely, several examples of processing obtaining the segmented image are provided above, the number of specific implementations is multiple, but is not limited to any one of the above.

In some embodiments, the segmented image can be extracted based on the mask region; in some other embodiments, the segmented image can be determined directly based on the image detection region, and the medical image as a whole in the image detection region can be used as the segmented image; with respect to the image to be processed determined based on the mask region, a small amount of background information and/or interference information is probably introduced.

In some embodiments, an obtaining method of the image to be processed may include the following operations.

The medical image is detected by using the second detection module to obtain the image detection region of the second target;

the image detection region of the second target is detected to obtain the outer contour information of the second target; and

the image to be processed is cut out according to the mask region corresponding to the outer contour information of the second target.

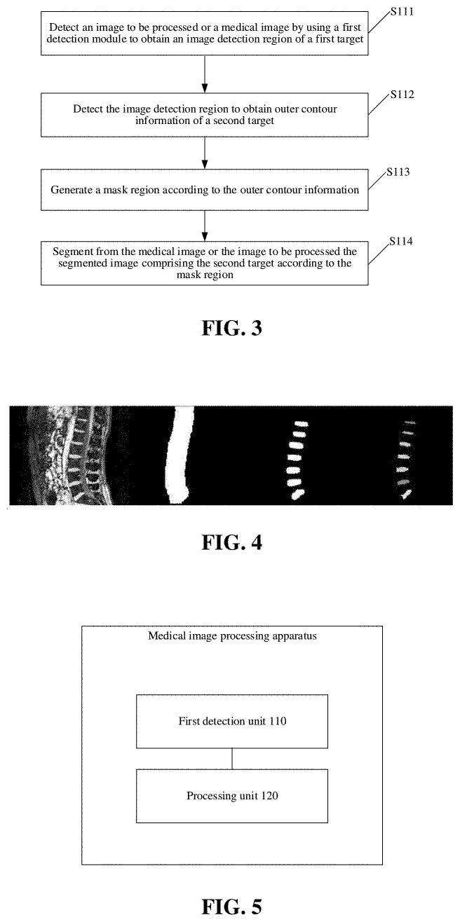

FIG. 4 includes sequentially from left to right: a lateral nuclear magnetic resonance image of the whole waist; a long-strip mask region of the vertebral column adjacent to the lateral nuclear magnetic resonance image and in the middle, a mask region of a single intervertebral disc, and the last schematic diagram of a segmented image of the single intervertebral disc.

In some embodiments, operation S120 includes the following operations.

The segmented image is processed to obtain the target feature map, wherein one target feature map corresponds to one first target; and the first diagnostic auxiliary information of the first target is obtained based on at least one of the image to be processed, the target feature map, or the segmented image.

Image processing is performed on the segmented image to obtain the target feature map, for example, the target feature map is obtained by means of convolution processing. The convolution processing may include: a convolution kernel which is preset and extracts features is convolved with image data of the image to be processed to extract the feature map. For example, the convolution processing of the fully connected convolutional network or the locally connected convolutional network in the neural network model is configured to outputting the target feature map.

In the embodiment, the first diagnostic auxiliary information of the first target is further obtained based on at least one of the image to be processed, the target feature map, or the segmented image to obtain the first diagnostic auxiliary information of the first target. For example, first identification information corresponding to the current target feature map is obtained according to the sorting of the first target corresponding to the target feature map in multiple first targets included in the image to be processed. It is convenient to a doctor to known by means of the first identification information which one of the first targets in the second target the current target feature map displays.

If the second target is the spine, the first target may be the intervertebral disc or vertebra; one intervertebral disc is provided between two adjacent vertebras. If the first target is the intervertebral disc, identifying can be performed according to adjacent vertebras. For example, the spine of a human may include: 12 thoracic vertebras, five lumbar vertebras, seven cervical vertebras, and one or more sacral vertebras. In the embodiments of the present disclosure, T represents the chest, L represents a lumbosacral portion, S represents a sacrum, and C represents a neck according to a medical naming rule; the vertebra is named as T1 and T2; the intervertebral disc is named as Tm1-m2, which represents that the intervertebral disc is an intervertebral disc between an m1-st thoracic vertebra and an m2rd thoracic vertebra. T12 may be configured to identifying the twelfth thoracic vertebra. Tm1-m2 and T12 are one of the first identification information of the first target. However, during specific implementation, other naming rules may further be adopted in the first identification information of the first target, for example, taking the second target as a reference as an example, sorting can be performed from top to bottom, and the sorting number is configured to identifying a corresponding vertebra or intervertebral disc.

In some embodiments, operation S120 further includes the following operations.

The first diagnostic auxiliary information of the corresponding first target is obtained directly according to the target feature map. For example, the size of the first target in different directions, for example, size information such as the length and thickness of the first target. The size information is one of attribute information of the first target. In some other embodiments, the attribute information may further include: shape information for describing the shape.

In some other embodiments, the first diagnostic auxiliary information further includes: different prompt information; for example, a feature different from that of a normal first target is generated for the first target, and the doctor can focus on viewing by generating alarm prompt information; the prompt information may further include: prompt information, generating the prompt information based on the attribute and a standard attribute of the first target. The prompt information is information automatically generated by the image processing device, and the medical personnel may need to further confirm the final diagnosis and treatment results. Therefore, the prompt information is the other prompt information for the medical personnel.

For example, the size of a certain first target displayed in the target feature map is oversize or undersize, and lesion may occur; the predicted conclusion of the lesion can be directly provided by means of the prompt information, and oversize or undersize information can also be prompted by means of the prompt information.

In conclusion, the types of the first diagnostic auxiliary information are multiple, and are not limited to any one of the above.

In some embodiments, operation S120 includes the following operations.

The first feature map is extracted from the segmented image by using a feature extraction layer of the first detection module;

At least one second feature map is generated by using a pooling layer of the first detection module based on the first feature map, wherein the size of the first feature map is different from that of the second feature map; and

The target feature map is obtained according to the second feature map.

In the embodiment, the first detection module may be the neural network model, the neural network model may include: multiple functional layers, and different functional layers have different functions. Each of the functional layers may include: an input layer, an intermediate layer, and an output layer; the input layer is configured to input data to be processed, the intermediate layer performs data processing, and the output layer outputs the processed result. Multiple neural nodes may be included between the input layer, the intermediate layer, and the output layer. Any one of the neural nodes of the next layer may be connected to all the neural nodes of the previous layer, and this belongs to an output fully connected neural network model. The neural node of the next layer is only connected to some of the neural nodes of the previous layer, and this belongs to a partially connected network. In the embodiment, the first detection module may be the partially connected network, so that a training time duration of the network can be reduced, the complexity of the network is reduced, and training efficiency is improved. The number of the intermediate layers may be one or more, and two adjacent intermediate layers are connected. Atomic layers of the described input layer, intermediate layer, and output layer, and one atomic layer includes multiple neural nodes that are provided in parallel; moreover, one functional layer includes multiple atomic layers.

In the embodiment, the extraction layer may be a convolutional layer, and the convolutional layer extracts features of different regions in the image to be processed by means of a convolution operation, for example, extracting a contour feature and/or a textural feature and the like.

A feature map is generated by means of feature extraction, that is, the first feature map. In order to reduce the subsequent calculated amount, the pooling layer is introduced in the embodiment, and the second feature map is generated by using down-sampling processing of the pooling layer. The number of features included in the second feature map is less than the original number of features included in the first feature map. For example, ½ down-sampling is performed on the first feature map, down-sampling a first feature map including N*M pixels to be a second feature map including (N/2)*(M/2) pixels. Down-sampling is performed on an adjacent domain during down-sampling. For example, down-sampling is performed on an adjacent domain of 2*2 consisting of four adjacent pixels to generate a pixel value of one pixel in the second feature map. For example, a maximal value, a minimal value, a mean value, or a mid-value in the 2*2 adjacent domain are output as the pixel value of the second feature map.

In the embodiment, the maximal value can be used as the pixel value of a corresponding pixel in the second feature map.

In this way, the data volume of the feature map is reduced by means of down-sampling, the subsequent processing is facilitated, and the speed can be improved; moreover, a receptive field of a single pixel is also improved. The number of pixels imaged or corresponded, in the original image, by one pixel of an image represented in the receptive field.

In some embodiments, multiple different sizes of second feature maps can be obtained by means of one or more pooling operations. For example, a first pooling operation is performed on the first feature map to obtain a first pooling feature map; a second pooling operation is performed on the first pooling feature map to obtain a second pooling feature map; a third pooling operation is performed on the second pooling feature map to obtain a third pooling feature map. In a similar fashion, when multiple pooling operations are performed again, pooling can be performed on the basis of the previous pooling operation, and different sizes of pooling feature maps are obtained finally. In the embodiments of the present disclosure, the pooling feature maps are called the second feature maps.

In the embodiment, three to five pooling operations can be performed on the first target feature map, so that the finally obtained second feature map has an enough receptive field, moreover, the data volume of the subsequent processing is also obviously reduced. For example, four pooling operations are performed based on the first feature map, and a fourth pooling feature map including the minimum number (i. e., the minimum size) of pixels is finally obtained.

Pooling parameters of different pooling operations may be different, for example, sampling coefficients of down-sampling are different, for example, the sampling coefficient of some pooling operations may be ½, and the sampling coefficient of some may be ¼. In the embodiment, the pooling parameters may be the same, so that model training of the first detection module can be simplified. The pooling layer also can correspond to the neural network model, so that the training of the neural network model can be simplified, and training efficiency of training the neural network model is improved.

In the embodiment, the target feature map is obtained according to the second feature map. For example, up-sampling is performed on the pooling feature map obtained by the last pooling to obtain the target feature map having the same image resolution as the input image to be processed. In some other embodiments, the image resolution of the target feature map can also be slightly lower than that of the image to be processed.

The pixel value in the feature map generated after the pooling operation substantively embodies an association relationship between adjacent pixels in the medical image.

In some embodiments, the processing the segmented image to obtain the target feature map includes the following operations.

Up-sampling is performed on the second feature map by using an up-sampling layer of the first detection module to obtain a third feature map;

the first feature map and the third feature map are fused by using a fusion layer of the first detection module to obtain a fusion feature map; or the third feature map and the second feature map different from the third feature map in dimension are fused to obtain a fusion feature map; and

the target feature map is output by using an output layer of the first detection module according to the fusion feature map.

The up-sampling layer may also consist of the neural network model, and the up-sampling can be performed on the second feature map; the pixel value can be increased by means of the up-sampling, and a sampling system of the up-sampling may be double or quadruple sampling. For example, a 16*16 third feature map can be generated for an 8*8 second feature map by means of the up-sampling of the up-sampling layer.

In the embodiment, a fusion layer is further included, and the fusion layer may also consist of the neural network model; the third feature map and the first feature map can be spliced, and the third feature map and the other second feature map different from the second feature map generating the third feature map can also be spliced.

For example, taking the 8*8 second feature map as an example, a 32*32 third feature map is obtained by means of the up-sampling, and the third feature map and the 32*32 second feature map are fused to obtain the fusion feature map.

In this case, the image resolution between two feature maps that the fusion feature map is obtained by means of fusion is the same, or it can said that the number of the included features or pixels is the same. For example, the feature map is represented in a matrix, and it can be considered that the number of the included features is the same or the number of the included pixels is the same.

The fusion feature map fuses the third feature map for the low-scale of second feature map, and therefore, has the enough receptive field, and fuses high-scale of the second feature map or the first feature map, and also covers sufficient detail information, so that the receptive field and information detail are taken into account for the fusion feature map, facilitating subsequently finally generating the target feature map to accurately express the attribute of the first target.

In the embodiment, the process of fusing the third feature map and the second feature map or fusing the third feature map and the first feature map may include: the fusing of the lengths is performed on feature values of multiple feature maps. For example, it is assumed that the image size of the third feature map is: S1*S2; the image size can be configured to describe the number of pixels or element formats included in a corresponding image. In some embodiments, each pixel or element of the third feature map further corresponds to: a feature length; if the feature length is L1. It is assumed that the image size of the second feature map to be fused is S1*S2, and the feature length of each pixel or element is: L2. Fusing such third feature map and second feature map may include: form a fusion image having S1*S2 of the image size; but the feature length of each pixel or element in the fusion image may be: L1+L2. Definitely, an example of the fusion between the feature maps is provided here, during specific implementation, there are multiple generation modes for the fusion feature map, but not limited to any one of the above.

The output layer can output, based on the probability, the most accurate fusion feature image in multiple fusion feature images as the target feature image.

The output layer may be: a softmax layer based on a softmax function; and may also be a sigmoid layer based on a sigmoid function. According to the output layer, the values of different fusion feature images can be mapped into the values between 0 to 1, and then the sum of the values may be 1, so as to satisfy a probability characteristic; the fusion feature map having the maximum probability value is selected after mapping and output as the target feature map.

In some embodiments, operation S120 includes one of the following operations.

First identification information of the first target corresponding to the target feature map is determined by combining the image to be processed and the segmented image;

attribute information of the first target is determined based on the target feature map; and

prompt information of the first target is determined based on the target feature map.

In this case, the first diagnostic auxiliary information may at least include the first identification information; in some other embodiments, in addition to the first identification information, the first diagnostic auxiliary information may further include: one or more of the attribute information and the prompt information. The attribute information may include: size information and/or shape information and the like.

Information content of the first identification information, the attribute information, and the prompt information may refer to the foregoing portion. Descriptions are not made herein in detail.

In some embodiments, the method further includes the following operations.

The second detection module and the first detection module are trained by using sample data;

network parameters of the second detection module and the first detection module are obtained by training by using sample data;

loss values of the second detection module and the first detection module in which network parameters are obtained are obtained based on a loss function; and

if the loss values are less than or equal to a preset value, the training of the second detection module and the first detection module is completed; or, if the loss values are greater than the preset value, the network parameters are optimized according to the loss values.

The sample data may include a sample image and data marked by the doctor for the second target and/or the first target. The network parameters of the second detection module and the first detection module can be obtained by training the sample data;

The network parameters may include: the weight and/or threshold influencing an input and an output between neural nodes. A product of the weight and the input and a weighted relationship between the product and the threshold influence the output of corresponding neural nodes.

It cannot be ensured that the corresponding second detection module and the first detection module have functions of accurately completing the segmentation of the image to be processed and the generation of the target feature map after the network parameters are obtained. Therefore, verification is performed in the embodiment. For example, by verifying the input of a verification image in data, the second detection module and the first detection module respectively obtain respective output, which is compared with marked data corresponding to the verification image, and the loss value can be calculated by using the loss function; the smaller the loss value is, the better the training result of the model is; when the loss value is smaller than the pre-set preset value, it can be considered that the optimization of the network parameter and the training of the model are completed. If the loss value is greater than the preset value, it can be considered that continuing to optimize is required, i. e., the model is required to continue to be trained till the loss value is smaller than or equal to the preset value, or the number of times of optimization reaches the upper limit of the number of times, the training of the model is stopped.

The loss function may be: a cross loss function or a DICE loss function and the like, but is not limited to any one during specific implementation.

In some embodiments, the optimizing the network parameters according to the loss values if the loss values are greater than the preset value includes the following operation.

The network parameters are updated by using a back propagation approach if the loss values are greater than the preset value.

The back propagation approach may be: traversing network paths from an output layer of one layer to an input layer. In this way, for a certain output node, only one traversal is performed on the path communicated with the output node when a reverse traversal is performed. Therefore, updating the network parameters by using the back propagation approach with respect to updating the network parameters by using a forward propagation approach can reduce repeated processing of the weight and/or threshold on the network path, and thus reducing the amount of processing and improving update efficiency. The forward propagation approach is to traverse the network paths in a direction from the input layer to the output layer to update the network parameter.

In some embodiments, the second detection module and the first detection module constitute one end-to-end model, and the end-to-end model relates to: directly inputting image data of the medical image that is required to be detected into the end-to-end model, a direction output is the desired output result, this model of directly outputting the result after processing input information by the model is called as the end-to-end model. However, the end-to-end model may consist of at least two sub-models that are connected to each other. The loss values of the second detection module and the first detection module may be respectively calculated, in this way, the second detection module and the first detection module respectively obtain respective loss value, and respectively optimize respective network parameter. However, the optimization approach when being in the subsequent use may accumulate the loss of the second detection module and the loss of the first detection module so as to result in low accuracy of the final output result. In view of the above, the calculating the loss values of the second detection module and the first detection module in which network parameters are obtained based on the loss function includes the following operation.

An end-to-end loss value which is input from the second detection module and output from the first detection module is calculated by using one loss function.

In the embodiment, one end-to-end loss value is calculated on the end-to-end model including the second detection module and the first detection module by directly using one loss function, network parameter optimization is performed on two models by using the end-to-end loss value, so that it can be ensured that a sufficiently accurate output result can be obtained when the model is applied online, that is, the sufficiently accurate target feature map and the first diagnostic auxiliary information.

It is assumed that the medical image in operation S110 is called as the current medical image, and it is assumed that the target feature map in operation S120 is called as the current target feature map; in some embodiments, the method further includes the following operations.

Second identification information of the current medical image is obtained;

a historical target feature map corresponding to a historical medical image is obtained according to the second identification information; the current target feature map and the historical target feature map of the same first target are compared, and second diagnostic auxiliary information is obtained;

and/or,

the first diagnostic auxiliary information corresponding to the historical medical image is obtained according to the second identification information; the first diagnostic auxiliary information of the current medical image and the first diagnostic auxiliary information corresponding to the historical medical image are compared, and third diagnostic auxiliary information is generated.

The second identification information may be an object identification of a diagnostic object, for example, taking a human diagnosis as an example, the second identification information may be: a hospitalizing number or a medical number of a patient.

Historical medical diagnostic information may be stored in a medical database. The target feature map and the first diagnostic auxiliary information are generated for the historical medical image by means of the medical image processing method of the present disclosure.

In the embodiment, the second diagnostic auxiliary information can be obtained by comparing the current medical image with the target feature map corresponding to the historical medical image, so as to help the medical personnel for intelligent comparison.

For example, in some embodiments, an animation sequence frame or a video is generated for the historical target feature map and the current target feature map of the same first target. The animation sequence frame or the video at least includes the historical feature map and the current target feature map, so as to dynamically represent the change of the target feature map of the same first target of the same diagnostic object by means of the animation sequence frame or the video, facilitating a user conveniently viewing the change and a change trend of the same first target by means of a visual image, and facilitating the medical personnel providing the diagnosis according to the change or change trend. The change of the same first target may be: one or more of a size change, a shape change and/or a texture change of the same first target.

For example, taking the intervertebral disc as the first target as an example, the second diagnostic auxiliary information may be text information and/or image information describing a size change or a size change trend of the first target. The image information may include a single picture, and may also include the aforementioned animation sequence frame or the video.

The animation sequence frame or the video including the historical feature map and the current target feature map is one of the second and first diagnostic auxiliary information. In some other embodiments, the second diagnostic auxiliary information may further be the text information.

The second diagnostic auxiliary information may further include: device evaluation information obtained by the medical image processing device according to the historical feature map and the current target feature map. For example, according to a shape change or a thickness change of a lumbar intervertebral disc, the device evaluation information of whether there is a lesion or the extent of the lesion is provided. The device evaluation information may be used as one of diagnostic auxiliary information of the doctor.

In some embodiments, the third diagnostic auxiliary information is generated by combining the first diagnostic auxiliary information corresponding to medical diagnostic information in different time points, and the third diagnostic auxiliary information may be generated based on a comparison difference between the first diagnostic auxiliary information generated by the medical images in different time points. For example, the third diagnostic information may include: conclusion information obtained by means of the change and a change trend of attribute information of the same first target. For example, the conclusion of whether a Dixon sequence size generated by a thoracic intervertebral disc T11-T12 during two diagnosis processes changes or changes in shape. In some embodiments, the third diagnostic information may further provide the change amount or change trend of the attribute information; certainly may also include the device evaluation information provided according to the change amount and/or change trend.

The target feature map and the first diagnostic auxiliary information corresponding to historical medical image information can be stored in a database of a medical system, and the target feature maps and the first diagnostic auxiliary information obtained in medical image information of the same patient in different times can be searched according to the second identification information, so that the device combines two or more adjacent medical image comprehensive information, and the comprehensive information may include one or more of the aforementioned target feature map, the first diagnostic auxiliary information, the second diagnostic auxiliary information, and the third diagnostic auxiliary information.

In some embodiments, the method further includes the following operations.

Links of the target feature map and/or the first diagnostic auxiliary information corresponding to a historical medical diagnosis image are established in an output page according to the second identification information while the target feature map and the first diagnostic auxiliary information of the current medical image are output after operation S130, and thus also facilitating the doctor conveniently obtaining the target feature map and/or the first diagnostic auxiliary information of the historical medical image according to the current needs by means of the links.

As shown in FIG. 5, the embodiments of the present disclosure provide a medical image processing apparatus, and the apparatus includes:

a first detection unit 110, configured to detect a medical image by using a first detection module to obtain first position information of a first target in a second target, wherein the second target includes at least two of the first targets; and

a processing unit 120, configured to segment the second target by using the first detection module according to the first position information to obtain a target feature map and first diagnostic auxiliary information of the first target.

In some embodiments, the first detection unit 110 and the processing unit 120 may be program units, the acquisition of the second position information of the second target, the extraction of the image to be processed, and the determination of the target feature map and the first diagnostic auxiliary information can be achieved after the program units are executed by a processor.

In some other embodiments, the first detection unit 110 and the processing unit 120 may be hardware or a combination of software and hardware. For example, the first detection unit 110 and the processing unit 120 may correspond to a field-programmable device or a complex programmable device. For another example, a butterfly module, the processing unit 120, and the processing unit 120 may correspond to an Application Specific Integrated Circuit (ASIC).

In some embodiments, the processing unit 120 is configured to perform a pixel-level segmentation on the second target by using the first detection module according to the first position information to obtain the target feature map and the first diagnostic auxiliary information.

In some embodiments, the apparatus further includes:

a second detection unit, configured to detect the medical image by using a second detection module to obtain the second position information of the second target in the medical image; and segment from the medical image an image to be processed comprising the second target according to the second position information; and

the first detection unit 110, configured to detect the medical image, obtain an image detection region where the second target is; detect the image detection region to obtain outer contour information of the second target; and generate a mask region according to the outer contour information.

In some embodiments, the processing unit 120 is configured to segment from the medical image the image to be processed according to the mask region.

In some embodiments, the first detection unit 110 is configured to detect the image to be processed or the medical image by using the first detection module to obtain an image detection region of the first target; detect the image detection region to obtain outer contour information of the first target; and generate a mask region according to the outer contour information, wherein the mask region is configured to segment the second target to obtain the first target.

In some embodiments, the processing unit 120 is configured to process the segmented image to obtain the target feature map, wherein one target feature map corresponds to one first target; and obtain the first diagnostic auxiliary information of the first target based on at least one of the image to be processed, the target feature map, or the segmented image.

In some embodiments, the processing unit 120 is configured to extract from the segmented image a first feature map by using a feature extraction layer of the first detection module; generate at least one second feature map by using a pooling layer of the first detection module based on the first feature map, wherein the scale of the first feature map is different from that of the second feature map; and obtain the target feature map according to the second feature map.

In some embodiments, the processing unit 120 is configured to perform up-sampling on the second feature map by using an up-sampling layer of the first detection module to obtain a third feature map; fuse the first feature map and the third feature map by using a fusion layer of the first detection module to obtain a fusion feature map; or fuse the third feature map and the second feature map different from the third feature map in scale to obtain a fusion feature map; and output the target feature map by using an output layer of the first detection module according to the fusion feature map.

In addition, the processing unit 120 is configured to execute at least one of the following operations.

First identification information of the first target corresponding to the target feature map is determined by combining the image to be processed and the segmented image;

attribute information of the first target is determined based on the target feature map; or prompt information generated on the basis of the attribute information of the first target based on the target feature map.

In some embodiments, the apparatus further includes:

a training unit, configured to obtain the second detection module and the first detection module by training by using sample data;

a calculation unit, configured to calculate loss values of the second detection module and the first detection module in which network parameters are obtained based on a loss function; and

an optimization unit, configured to optimize the network parameters according to the loss values if the loss values are greater than a preset value; or a training unit, further configured to complete the training of the second detection module and the first detection module if the loss values are less than or equal to the preset value.

In some embodiments, the optimization unit is configured to update the network parameter by using a back propagation approach if the loss values are greater than the preset value.

In some embodiments, the calculation unit is configured to calculate an end-to-end loss value which is input from the second detection module and output from the first detection module by using one loss function.

In some embodiments, the second target is a spine; and

the first target is: an intervertebral disc.

Several specific examples are provided below by combining the aforementioned any embodiment:

### Example 1

Firstly, the intervertebral disc is detected and positioned by using a deep learning model to obtain position information of each intervertebral disc, for example, a central coordinate of each intervertebral disc is obtained, and which intervertebral disc the intervertebral disc is is marked (that is, marking two of which vertebrae the intervertebral disc is located between, for example, between a thoracic vertebra T12 and a lumbar vertebra L1). The deep learning model may include the aforementioned neural network model.

By combining the position information, detected in the previous operation, of the intervertebral disc, a pixel-level segmentation is performed on the intervertebral disc by using the deep learning model, so as to obtain information such as complete boundary, shape, and volume of the intervertebral disc for assisting the doctor to perform the diagnosis.

A deep learning framework in the example is a full-automatic end-to-end solution, complete intervertebral disc detection and segmentation results can be output by inputting the medical image.

Specifically, the method provided in the example may include the following operations.

Firstly, a two-dimensional image in a Dixon sequence of the intervertebral disc is pre-processed, and re-sampling is performed on the image, and thus being equivalent to replicating the image of the Dixon sequence; moreover, the original Dixon sequence can be used for archiving or backup use.

The neural network model having a detection function is used for detecting the position of the intervertebral disc to obtain a detection frame specifying the intervertebral disc and the mask region located in the detection frame, and the mask region is used for segmenting the intervertebral disc in the next operation so as to obtain a single intervertebral disc.

A convolution kernel may have a bigger receptive field by using a full convolutional neural network model (for example, a U-Net) by means of down-sampling.

A feature map in which convolution processing is performed is restored to the size of the original image by means of up-sampling, and a segmentation result is obtained by means of a softmax layer. The segmentation result may include: the target feature map and the first diagnostic auxiliary information.

A fusion layer fused by different scales of target feature maps can be added into the neural network model to improve segmentation accuracy. Synchronizing the fusion of different scales of images, so that an image including a bigger receptive field and an image including bigger image original details are fused together, so as to obtain an image having the bigger receptive field and also including sufficient original details.

A cross-entropy loss function is used in the loss function, the segmentation result predicted by a network is compared with the marking of the doctor by using the loss function, and the parameter of the model is updated by means of back propagation.

The mask region obtained by detecting the intervertebral disc is segmented for training aiding, most useless backgrounds are excluded, so that the network can focus on the region around the intervertebral disc and segmentation accuracy can be effectively improved.

The detection of the intervertebral disc and the acquisition of the mask region, and the pixel-level segmentation of the intervertebral disc.

As shown in FIG. 4, an original medical image, a vertebral column segmentation result, the mask region of specified intervertebral discs (seven between T11-S1) obtained by a detection network and the segmentation result of the intervertebral disc are respectively comprised from left to right.

The detection and segmentation of the intervertebral disc may respectively include the following operations.

A segmentation result of a vertebral column portion is obtained by using a segmentation algorithm according to the input Dixon sequence, and the interference of other portions is excluded. The operations specifically include the following operations. The Dixon sequence is input into the detection network, and the specific position of the intervertebral disc is detected and a rough mask region is generated for segmenting by using the limit of the segmentation result of the vertebral column; segmenting is performed based on a two-dimensional image of a full convolutional network. Each frame of image in the Dixon sequence is separately segmented, and then is integrated together to obtain a complete segmentation result.

A structure based on an FCN or a U-Net and an improved model of the FCN and U-Net is adopted in a network structure. Convolution of different layers and four pooling operations are performed on the original image, a 128*128 image is down-sampled to form feature maps having sizes of 64*64, 32*32, 16*16, and 8*8. In this way, convolution kernels having the same size can have a more and more bigger receptive field. After the feature map of the intervertebral disc is obtained, the original resolution is restored by means of a deconvolution or an interpolation method. Because the resolution after the down-sampling is gradually decreased, lots of detail information is lost, and then different scales of feature maps can be fused, for example, a short connected connection is added between down-sampling and up-sampling layers having the same resolution, so that the detail information is gradually restored during an up-sampling process.

The segmentation result is obtained by means of the softmax layer, and is compared with the marking of the doctor, and other loss functions such as a cross-entropy loss or DICE are calculated.

When the loss value is calculated, the loss, to the mask region of the intervertebral disc, of the detection network is only calculated, and thus lots of irrelevant backgrounds can be neglected, so that the network can focus on the region around the intervertebral disc and the segmentation accuracy is improved. The parameter of the model is updated by means of back propagation, and the model is iteratively optimized till the model converges or reaches the maximum number of iterations.

A vertebral column segmentation is used as a limit, a detection algorithm is combined, and the algorithm has stronger stability. An accurate segmentation is performed after the detection, the interference is excluded, and the segmentation result is more accurate.

The vertebral column segmentation is used as the limit, and the detection algorithm is combined. The algorithm has stronger stability.

The accurate segmentation is performed after the detection, the interference is excluded, and the segmentation result is more accurate.

the segmentation result is more accurate, and thus parameters such as the volume obtained by calculating thereby are also more accurate. The doctor can be helped to make a diagnosis in a better way.

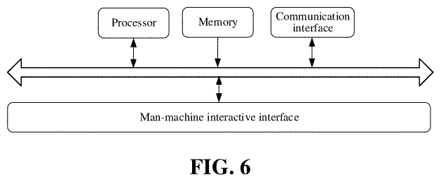

As shown in FIG. 6, the embodiments of the present disclosure provide an image processing device, including:

a memory, configured to store information; and

a processor, connected to the memory, and configured to execute computer executable instructions stored on the memory to implement the image processing methods provided in the aforementioned one or more technical solutions, for example, the methods as shown in FIG. 1, FIG. 2 and/or FIG. 3.

The memory may be different types of memories, may be a random access memory, a Read-only Memory (ROM), a flash memory and the like. The memory can be used for information storage, for example, storing the computer executable instructions and the like. The computer executable instructions may be different program instructions, for example, a target program instruction and/or a source program instruction and the like.

The processor may be different types of processors, for example, a central processing unit, a microprocessor, a digital signal processor, a programmable array, a digital signal processor, an application-specific integrated circuit, or an image processor or the like.

The processor can be connected to the memory by means of a bus. The bus may be an integrated circuit bus and the like.

In some embodiments, a terminal device may further include: a communication interface, and the communication interface may include: a network interface, for example, a local area network interface, a transceiving antenna and the like. The communication interface is also connected to the processor, and can be used for information receiving and transmitting.

In some embodiments, the terminal device further includes a man-machine interactive interface, for example, the man-machine interactive interface may include different input/output devices, for example, a keyboard, a touch screen and the like.

The embodiments of the present disclosure provide a computer storage medium, and the computer storage medium stores computer executable codes; after the computer executable codes are executed, the image processing methods provided in the aforementioned one or more technical solutions can be implemented, for example, one or more of the methods shown in FIG. 1, FIG. 2, and FIG. 3 can be implemented.

The storage medium includes: various media capable of storing program codes such as a portable storage device, a ROM, a Random Access Memory (RAM), a magnetic disk, or an optical disk. The storage medium may be a non-instantaneous storage medium.

The embodiments of the present disclosure provide a computer program product, and the program product includes computer executable instructions; after the computer executable instructions are executed, the image processing methods provided in the aforementioned one or more technical solutions can be implemented, for example, one or more of the methods shown in FIG. 1, FIG. 2, and FIG. 3 can be implemented.

The computer executable instructions included in the computer program product in the embodiment may include: an application program, a software development kit, a plugin or a patch or the like.

It should be understood that the disclosed device and method in the embodiments provided in the present disclosure may be implemented by other modes. The device embodiments described above are merely exemplary. For example, the unit division is merely logical function division and may be actually implemented by other division modes. For example, a plurality of units or components may be combined or integrated into another system, or some features may be ignored or not performed. In addition, the displayed or discussed mutual couplings or direct couplings or communication connections among the components may be implemented by means of some interfaces. The indirect couplings or communication connections between the devices or units may be implemented in electronic, mechanical, or other forms.

The units described as separate components may or may not be physically separate, and the components displayed as units may or may not be physical units, may be located at one position, or may be distributed on a plurality of network units. A part of or all of the units may be selected according to actual needs to achieve the objectives of the solutions of the embodiments.

In addition, the functional units in the embodiments of the present disclosure may be integrated into one processing unit, or each of the units may exist as an independent unit, or two or more units are integrated into one unit, and the integrated unit may be implemented in the form of hardware, or may also be implemented in the form of a hardware and software functional unit.

A person of ordinary skill in the art may understand that: all or some operations of implementing the forgoing embodiments of the method may be achieved by a program by instructing related hardware; the foregoing program may be stored in a computer-readable storage medium; when the program is executed, operations including the foregoing embodiments of the method are performed; moreover, the foregoing storage medium includes various media capable of storing the program codes such as the portable storage device, the ROM, the RAM, the magnetic disk, or the optical disk.

The descriptions above are only specific implementations of the present disclosure. However, the scope of protection of the present disclosure is not limited thereto. Within the technical scope disclosed by the present disclosure, any variation or substitution that can be easily conceived of by those skilled in the art should all fall within the scope of protection of the present disclosure. Therefore, the scope of protection of the present disclosure should be defined by the scope of protection of the claims.

1. A medical image processing method, comprising:
detecting a medical image by using a first neural network to obtain first position information of a first target in a second target, wherein the second target comprises at least two of the first targets; and
segmenting the second target by using the first neural network according to the first position information to obtain a target feature map of the first target and a first diagnostic auxiliary information of the first target.

2. The method according to claim 1, wherein
the segmenting the second target by using the first neural network according to the first position information to obtain the target feature map of the first target and the first diagnostic auxiliary information of the first target comprises:
performing a pixel-level segmentation on the second target by using the first neural network according to the first position information to obtain the target feature map and the first diagnostic auxiliary information.

3. The method according to claim 1, further comprising:
detecting the medical image by using a second neural network to obtain second position information of the second target in the medical image; and
segmenting from the medical image an image to be processed comprising the second target according to the second position information; and
the detecting the medical image by using the first neural network to obtain the first position information of the first target in the second target comprising:
detecting the image to be processed by using the first neural network to obtain the first position information.

4. The method according to claim 3, wherein
the detecting the medical image by using the first neural network to obtain the first position information of the first target in the second target comprises:
detecting the image to be processed or the medical image by using the first neural network to obtain an image detection region of the first target;
detecting the image detection region to obtain outer contour information of the first target; and
generating a mask region according to the outer contour information, wherein the mask region is configured to segment the second target to obtain a segmented image of the first target.

5. The method according to claim 4, wherein
the segmenting the second target by using the first neural network according to the first position information to obtain a target feature map of the first target and a first diagnostic auxiliary information of the first target comprises:
segmenting the second target according to the mask region to obtain a segmented image of the first target;
processing the segmented image to obtain the target feature map, wherein one target feature map corresponds to one first target; and
obtaining the first diagnostic auxiliary information of the first target based on at least one of the image to be processed, the target feature map, or the segmented image.

6. The method according to claim 5, wherein
the processing the segmented image to obtain the target feature map comprises:
extracting from the segmented image a first feature map by using a feature extraction layer of the first neural network;
generating at least one second feature map by using a pooling layer of the first neural network based on the first feature map, wherein a scale of the first feature map is different from a scale of the second feature map; and
obtaining the target feature map according to the second feature map.

7. The method according to claim 6, wherein
the processing the segmented image to obtain the target feature map comprises:
performing up-sampling on the second feature map by using an up-sampling layer of the first neural network to obtain a third feature map;
fusing the first feature map and the third feature map by using a fusion layer of the first neural network to obtain a fusion feature map; or fusing the third feature map and the second feature map different from the third feature map in scale to obtain a fusion feature map; and
outputting the target feature map by using an output layer of the first neural network according to the fusion feature map.

8. The method according to claim 6, wherein
the obtaining the first diagnostic auxiliary information of the first target based on at least one of the image to be processed, the target feature map, or the segmented image comprises at least one of the following:
determining first identification information of the first target corresponding to the target feature map by combining the image to be processed and the segmented image;
determining attribute information of the first target based on the target feature map; or
determining prompt information generated on the basis of the attribute information of the first target based on the target feature map.

9. The method according to claim 3, further comprising:
obtaining the second neural network and the first neural network by training by using sample data; and
calculating loss values of the second neural network and the first neural network in which network parameters are obtained based on a loss function; and
responsive to the loss values being less than or equal to a preset value, completing the training of the second neural network and the first neural network; or, responsive to the loss values being greater than the preset value, optimizing the network parameters according to the loss values.

10. The method according to claim 9, wherein
the responsive to the loss values being greater than the preset value, optimizing the network parameters according to the loss values comprises:
responsive to the loss values being greater than the preset value, updating the network parameters by using a back propagation approach.

11. The method according to claim 9, wherein
the calculating the loss values of the second neural network and the first neural network in which network parameters are obtained based on the loss function comprises:
calculating, by using one loss function, an end-to-end loss value which is input from the second neural network and output from the first neural network.

12. The method according to claim 1, further comprising:
obtaining the medical image.

13. The method according to claim 1, wherein
the second target is a spine; and
the first target is: an intervertebral disc.

14. The method according to claim 1, wherein the method is performed by an image processing device;
the method further comprises:
displaying, on a screen of the image processing device, the target feature map of the first target and the first diagnostic auxiliary information of the first target.

15. An image processing device, comprising:
a memory, configured to store information;
a processor, connected to the memory, and configured to execute computer executable instructions stored on the memory to implement the following operations:
detecting a medical image by using a first neural network to obtain first position information of a first target in a second target, wherein the second target comprises at least two of the first targets; and
segmenting the second target by using the first neural network according to the first position information to obtain a target feature map of the first target and a first diagnostic auxiliary information of the first target.

16. The device according to claim 15, wherein the segmenting the second target by using the first neural network according to the first position information to obtain the target feature map of the first target and the first diagnostic auxiliary information of the first target comprises:
performing a pixel-level segmentation on the second target by the first neural network according to the first position information to obtain the target feature map and the first diagnostic auxiliary information.

17. The device according to claim 15, further comprising:
detecting the medical image by using a second neural network to obtain second position information of the second target in the medical image; and
segmenting from the medical image an image to be processed comprising the second target according to the second position information; and
the detecting the medical image by using the first neural network to obtain the first position information of the first target in the second target comprising:
detecting the image to be processed by using the first neural network to obtain the first position information.

18. The device according to claim 17, wherein
the detecting the medical image by using the first neural network to obtain the first position information of the first target in the second target comprises:
detecting the image to be processed or the medical image by using the first neural network to obtain an image detection region of the first target;
detecting the image detection region to obtain outer contour information of the first target; and
generating a mask region according to the outer contour information, wherein the mask region is configured to segment the second target to obtain the first target.

19. The device according to claim 17, wherein
the segmenting the second target by using the first neural network according to the first position information to obtain a target feature map of the first target and a first diagnostic auxiliary information of the first target comprises:
segmenting the second target according to a mask region to obtain a segmented image of the first target;
processing the segmented image to obtain the target feature map, wherein one target feature map corresponds to one first target; and
obtaining the first diagnostic auxiliary information of the first target based on at least one of the image to be processed, the target feature map, or the segmented image.

20. A non-transitory computer storage medium, configured to store computer-readable instructions, wherein execution of the instructions by a processor causes the processor to perform:
detecting a medical image by using a first neural network to obtain first position information of a first target in a second target, wherein the second target comprises at least two of the first targets; and
segmenting the second target by using the first neural network according to the first position information to obtain a target feature map of the first target and a first diagnostic auxiliary information of the first target.
